(19) United States
(12) Patent Application Publication
(10) Pub. No.: US 2024/0180743 A1
(43) Pub. Date: Jun. 6, 2024

(54) METHODS, DEVICES, AND SYSTEMS FOR MAMMALIAN BODY TEMPERATURE MANIPULATION

(71) Applicant: ARTERIA TECHNOLOGY, INC., Boca Raton, FL (US)

(72) Inventors: Theodora Warlick, Boca Raton, FL (US); Austin Waggoner, Longmont, CO (US); Scott Greenberg, Boca Raton, FL (US); Craig Gile, Boca Raton, FL (US)

(21) Appl. No.: 18/285,280

(22) PCT Filed: Apr. 4, 2022
(86) PCT No.: PCT/US2022/023255

§ 371 Date: Oct. 2, 2023

Related U.S. Application Data
(60) Provisional application No. 63/170,142, filed on Apr. 2, 2021.

(51) Int. Cl. A61F 7/00

(52) U.S. Cl. CPC A61F 7/0053; A61F 7/0085; A61F 2007/0036; A61F 2007/006; A61F 2007/0064; A61F 2007/0069; A61F 2007/0076; A61F 2007/0094; A61F 2007/0096

(57) ABSTRACT

Methods, devices, and systems for regulating mammalian body temperature are provided. In practicing the methods, devices including glabrous skin contact devices, heat exchange devices and devices comprising both glabrous skin contact devices and heat exchange devices are used to contact the glabrous skin of a mammal with a warming or cooling and with optional negative pressure thereby regulating the mammal's body temperature. The devices, methods, and systems can find uses in athletic, medical, and occupational health settings.

### PRIORITY

This application claims priority to provisional No. 63/170,142 (entitled “Methods, Devices, and Systems for Mammalian Body Temperature Manipulation” and filed on Apr. 2, 2021), which is hereby incorporated by reference in its entirety.

### FIELD OF THE INVENTION

Embodiments of these inventions relate generally to the field of mammalian body temperature regulation.

### BACKGROUND OF THE INVENTION

While it is known that mammalian body temperature can be modulated by warming or cooling glabrous skin surfaces such as the soles of the feet, palms of the hand, etc., practical, and effective devices remain elusive. U.S. Pat. No. 8,117,826 describes devices for encasing a bodily appendage such as an arm or hand such that the appendage is sealed in a chamber where heat or cold is applied with negative pressure. These devices require particularly good seals to ensure that the proper application of negative pressure is created. Creating too much negative pressure can have the deleterious effect of limiting blood flow. Similarly, U.S. Pat. No. 8,066,752 describes devices that also encase appendages and require complicated seals for the proper creation of negative pressure. Even the wearable cooling system with a palm interface without negative pressure described by authors Grahn, Makam, and Heller in their article entitled “A method to reduce heat strain while clad in encapsulating outerwear” and published in the Journal of Occupational and Environmental Hygiene, April, 2018 shows the need for further optimization of these devices. Thus, there is a need for improved devices that can regulate body temperature and be useful in athletic, medical, and occupational situations.

### SUMMARY OF THE INVENTION

In some embodiments, a body temperature modifying device comprises a heat exchange unit connected by at least one tubing unit to a glabrous skin contact unit, wherein the glabrous skin contact unit comprises a housing enclosing a bladder disposed in an interior region of the glabrous skin contact unit, and wherein the housing has a molded bottom surface and a contoured upper surface. Additionally, in some embodiments, the molded bottom surface includes a protuberance that protrudes into an interior region of the glabrous skin contact unit. In other embodiments, the molded bottom surface has a Young's Modulus that is greater than a Young's Modulus of the contoured upper surface. In still other embodiments, the glabrous skin contact unit further comprises a means for creating negative pressure and in some embodiments, the means for creating negative pressure creates from about 0.5 mm-Hg to about 50 mm-Hg of pressure. Also, in some embodiments, the means for creating negative pressure can be selectively operated.

In some embodiments, a method of manipulating mammalian body temperature comprises the steps of subjecting a glabrous skin surface to a body temperature modifying device comprising a heat exchange unit connected by at least one tubing unit to a glabrous skin contact unit, wherein the glabrous skin contact unit comprises a housing enclosing a bladder disposed in an interior region of the glabrous skin contact unit, and wherein the housing has a molded bottom surface and a contoured upper surface, and operating the heat exchange unit with a temperature transfer fluid.

In some embodiments, a method of improving athletic performance comprises the steps of subjecting a glabrous skin surface to a glabrous skin contact device, wherein the glabrous skin contact unit comprises a molded bottom surface, a contoured upper surface, and a bladder disposed in an interior region of the glabrous skin contact unit, and wherein the glabrous skin surface is subjected to the glabrous skin contact device for a temperature adjustment time of at least fifteen seconds.

### BRIEF DESCRIPTION OF THE DRAWINGS

For clarity and understanding, some embodiments of the inventions are described in more detail in the appended drawings. These drawings should not, however, be considered as limiting the scope of the inventions and the inventions include any number of other equally useful embodiments.

FIG. 1 is a perspective view of an embodiment of the glabrous skin contact device.

FIG. 2 is a cross-sectional view of an embodiment of the glabrous skin contact device having a bladder disposed therein.

FIG. 3 is a cross-sectional view of an embodiment of the glabrous skin contact device having an elastic panel disposed therein.

FIG. 4 is a perspective view of an embodiment of the heat exchange device.

FIG. 5 is an exploded view of an embodiment of the control unit portion of the heat exchange device.

FIG. 6 is an exploded view of an embodiment of the control unit portion of the heat exchange device.

FIG. 7 is an exploded view of an embodiment of the gasket in the heat exchange device.

FIG. 8 is a perspective view of an embodiment of the heat exchanger.

FIG. 9 is a perspective view of an embodiment of the bladder of the glabrous skin contact device.

FIG. 10 is a perspective view of an embodiment of a glabrous skin surface positioned in the glabrous skin contact device.

FIG. 11 is a perspective view of an embodiment of the bladder removed from the housing of the glabrous skin contact device.

### DETAILED DESCRIPTION OF THE INVENTION

To provide a simple, effective apparatus and various methods of use for manipulating body temperature, some embodiments include a glabrous skin contact device, a heat exchange device, and a body temperature modifying device that includes both a glabrous skin contact unit and a heat exchange unit connected by at least one tubing unit. Some embodiments can be used for reducing body temperature, while other embodiments can be used for increasing body temperature. The glabrous skin to which the apparatus and methods can be applied may belong to a mammal such as a human, a dog, a cat, a rodent, etc.

In one embodiment, a body temperature modifying device comprises a glabrous skin contact unit that can contain an appendage of a mammal such as a hand or a foot and the glabrous skin contact unit houses a bladder through which a warm or cool temperature transfer fluid provided by a heat exchange unit flows to provide body temperature raising or lowering. In some embodiments, negative pressure can be optionally or intermittently applied to the glabrous skin contact unit to facilitate body temperature manipulation. The application of negative pressure can be selectively controlled by a user of the device or can be applied in response to a sensor such as a vasoconstriction sensor, a vasodilation sensor, or a body temperature sensor.

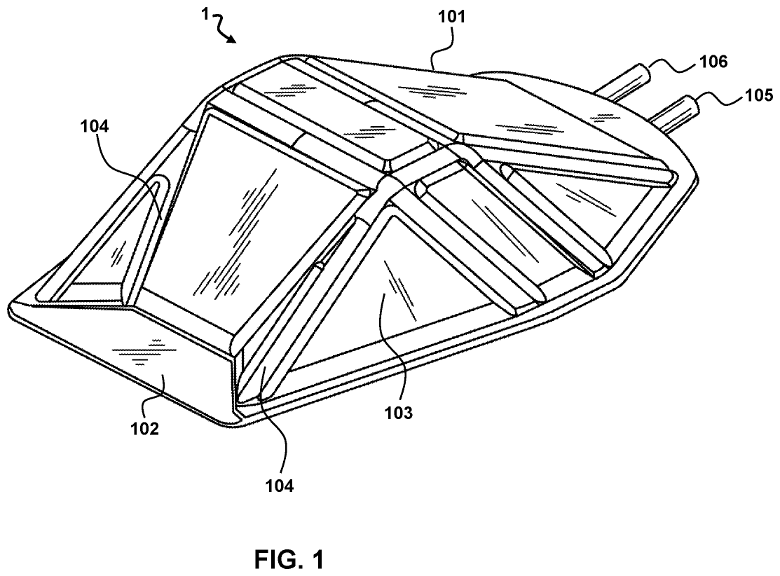

FIG. 1 is a perspective view of an embodiment of the glabrous skin contact device (1). The glabrous skin contact device (1) comprises a housing (101) enclosing a bladder (201—described below) disposed in an internal region of the housing, wherein the housing (101) comprises a molded bottom surface (102) and a contoured upper surface (103). As used herein, the term “molded bottom surface” refers to a non-flat, unitary section of the device that has been shaped to facilitate body temperature modification. As used herein, the term “contoured upper surface” refers to a non-flat, unitary section that has been shaped to house an appendage of a mammal.

In some embodiments, the housing (101) provides sufficient insulating capacity such that parasitic heat loss from the bladder to the environment is minimized. Without this insulating capacity, efficiency and efficacy of the glabrous skin contact unit are diminished.

In some embodiments, the molded bottom surface (102) has a Young's Modulus that is greater than the contoured upper surface (103). In other embodiments, the Young's Modulus of the molded bottom surface (102) is from about 0.75 Pascal (GPa) to about 4.15 GPa. In some embodiments, the Young's Modulus of the molded bottom surface (102) is from about 0.75 GPa, 1 GPa, 1.25 GPa, 1.5 GPa, 1.75 GPa, 2 GPa, 2.25 GPa, 2.50 GPa, 2.75 GPa, 3 GPa, 3.25 GPa, 3.5 GPa, 3.75 GPa, or 4 GPa to about 1 GPa, 1.25 GPa, 1.5 GPa, 1.75 GPa, 2 GPa, 2.25 GPa, 2.50 GPa, 2.75 GPa, 3 GPa, 3.25 GPa, 3.5 GPa, 3.75 GPa, 4 GPa, or 4.15 GPa. Those of skill in the art will appreciate various ways to obtain the measurement of Young's Modulus and any conventional extensometer can be used to measure the Young's Modulus of a material. Without wishing to be bound by any theory, it is thought that having materials with Young's Modulus in this range for the molded bottom surface (102) provides sufficient strength for the glabrous skin contact device to house the bladder and optionally exert negative pressure without collapsing the protuberance (202—described below) for enhanced body temperature manipulation.

While a molded bottom surface with a Young's Modulus in the range described above is preferable in many embodiments, there are other embodiments where the molded bottom surface has a Young's Modulus of up to 300 GPa because the molded bottom surface integrates a heat exchanger such that the bladder does not sit on top of the molded bottom surface.

The glabrous skin contact device (1) further comprises an inlet port (105) and an outlet port (106) both adhered to an upper interior surface of the molded bottom surface (102). The glabrous skin contact device (1) also comprises at least one opening to receive a glabrous skin appendage and the contoured upper surface (103) comprises at least two passive closure features (104) located on at least two edges of the at least one opening. The passive closure features allow the opening to enlarge sufficiently to receive the glabrous skin appendage and then close around the appendage in a way that does not restrict blood flow to the appendage and is comfortable for the user of the device. In some embodiments, the housing comprises a material with sufficient elasticity that closure features are not necessary.

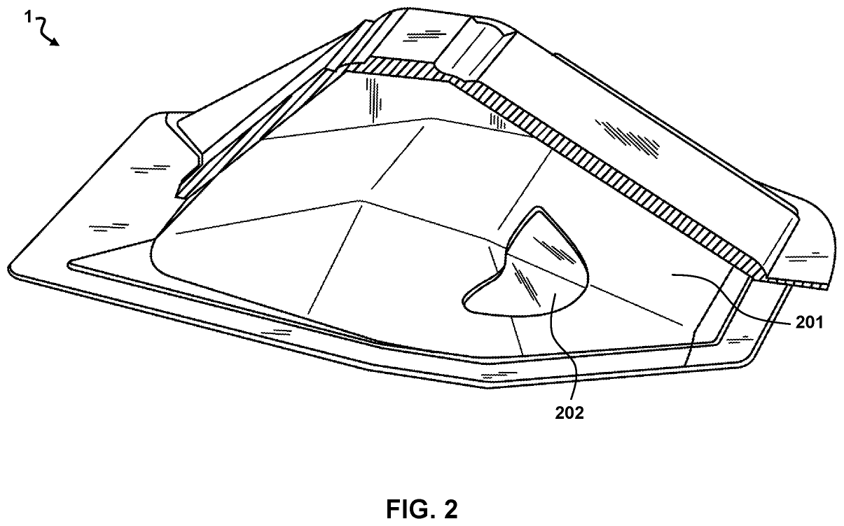

FIG. 2 is a cross-sectional view of an embodiment of the glabrous skin contact device (1) having a bladder (201) disposed therein with a protuberance (202) that protrudes into an interior region of the glabrous skin contact device. In some embodiments, the bladder (201) is made from at least one material having a thermal conductivity of from about 0.1 to about 0.5 watts per meter-kelvin (W·m−1·K−1). Materials with thermal conductivity in this range offer sufficient heat transfer so that a temperature transfer fluid in the bladder will provide warming or cooling to the glabrous skin of a user, thereby causing an increase or decrease in body temperature. Suitable materials in some embodiments include, but are not limited to polyurethanes, polyvinyl chlorides, nylons, thermoplastic polyurethanes, polyethylene terephthalates, and the like. In some embodiments, the bladder is a patterned perfusion pad. These materials also provide flexibility that allow the bladder to conform to the contour of the glabrous skin of the appendage and to conform to the contour of the protuberance thus optimizing contact between the bladder and the glabrous skin. Additionally, these materials provide sufficient strength to remain inflated under negative or positive pressure.

In some embodiments, the bladder is welded with a design pattern to equalize the distribution of temperature transfer fluid throughout the contact surface of the bladder while maintaining a uniform temperature of the temperature transfer fluid.

In some embodiments, the bladder and tubing have a volume of temperature transfer fluid of from about 75 ml to about 125 ml. In some embodiments, the volume is from about 75 ml, 80 ml, 85 ml, 90 ml, 95 ml, 100 ml, 105 ml, 110 ml, 115 ml, or 120 ml to about 80 ml, 85 ml, 90 ml, 95 ml, 100 ml, 105 ml, 110 ml, 115 ml, 120 ml, or 125 ml. In some embodiments, the bladder is disposable.

In some embodiments, the protuberance (202) has a height of from about 1 mm at its junction with the molded bottom surface to about 100 mm at its apex. In some embodiments, the protuberance has a width of from about 12 cm to about 15 cm and a length along the long axis of the molded bottom surface of from about 18 cm to about 23 cm. In other embodiments, the protuberance (202) has a forward slope of from about 5 degrees to about 7 degrees leading to an apex. While in still other embodiments, the protuberance (202) has a rear slope of from about 20 degrees to about 25 degrees leading away from the apex. In some embodiments, the size and shape of the protuberance (202) ensures that there is sufficient contact between the glabrous skin of a user and the bladder (201) and limits the tendency for a user to grip the bladder. In some embodiments, the protuberance is symmetrical front to back.

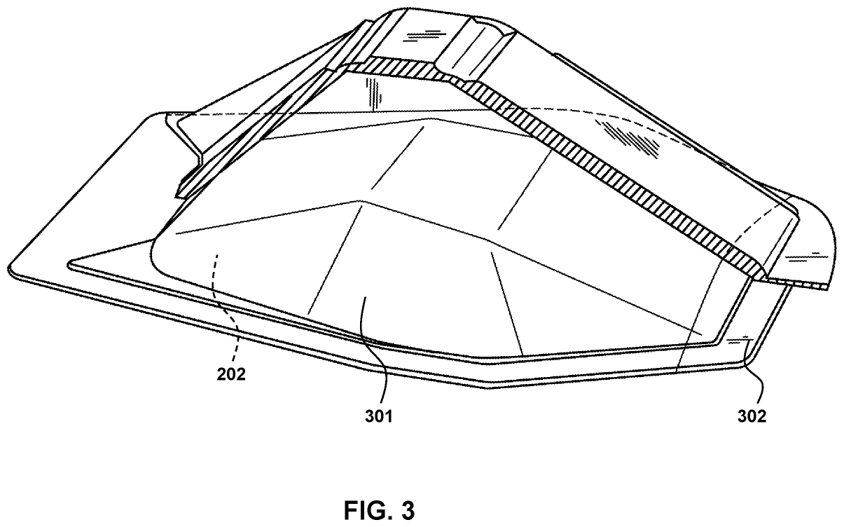

FIG. 3 is a cross-sectional view of an embodiment of the glabrous skin contact device having a panel (301) disposed therein. In some embodiments, the panel (301) is disposed in an interior region of the glabrous skin contact device and the panel (301) is adhered to an upper interior surface of at least one edge of the molded bottom surface (302). In some embodiments, the panel (301) is adhered to the upper interior surface of at least one edge of the molded bottom surface (302) so that the panel (301) creates a pocket for the bladder. In some embodiments, the panel completely covers the bladder while in other embodiments, the panel partially covers the bladder. In some embodiments, the panel (301) can be formed from materials such as, but not limited to, woven or non-woven elastics; nylon, polyester, latex free, cotton, elastics, or combination thereof. In some embodiments, the panel is a means for stabilizing the bladder so that it does not shift or move during use.

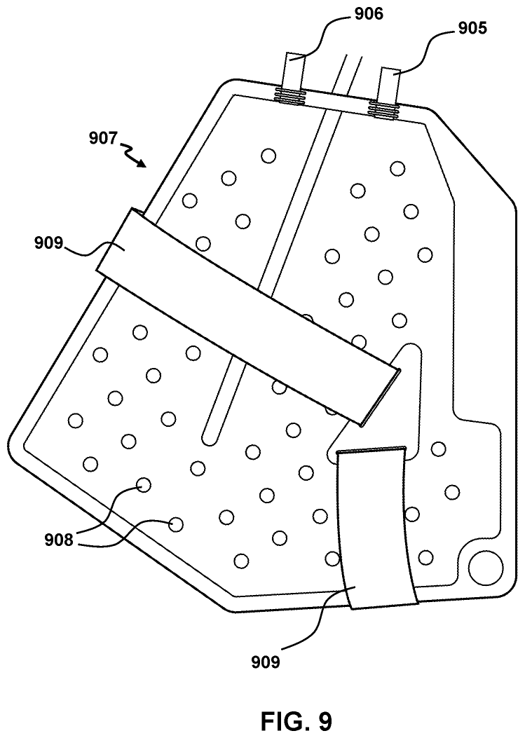

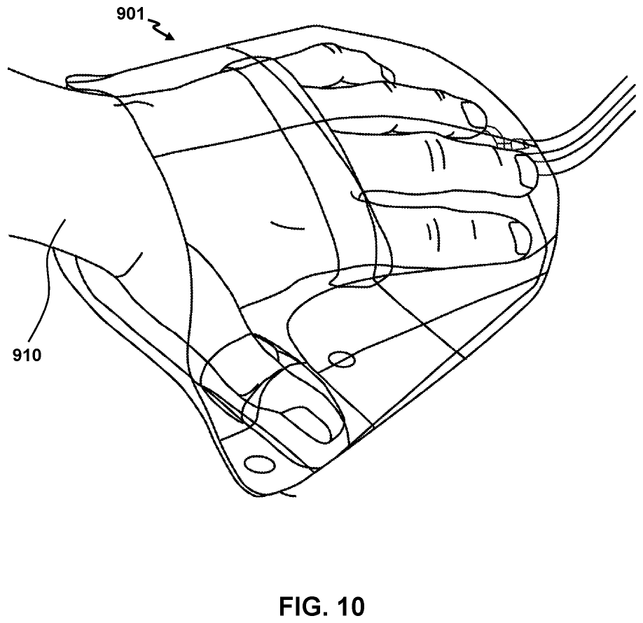

FIG. 9 is a perspective view of an embodiment of the bladder (907) of the glabrous skin contact device. In some embodiments, the bladder (907) comprises a top surface that includes a plurality of protruding spots (908). The protruding spots (908) protrude out of the top surface of the bladder (907) to provide cooling or heating to the glabrous skin surface. Further, the bladder (907) comprises an inlet port (905) and an outlet port (906). In some embodiments, the bladder (907) comprises a top surface having straps (909) to receive the glabrous skin surface.

FIG. 10 is a perspective view of an embodiment of a glabrous skin surface (910) positioned in the housing (901) of the glabrous skin contact device. The glabrous skin surface (910) is positioned snugly in the elastic straps (909) to yield a comfortable yet tight fit. FIG. 10 shows the glabrous skin surface (910) to be the palm of a hand but is not limited to that. For example, the glabrous skin surface could also be the sole of the foot.

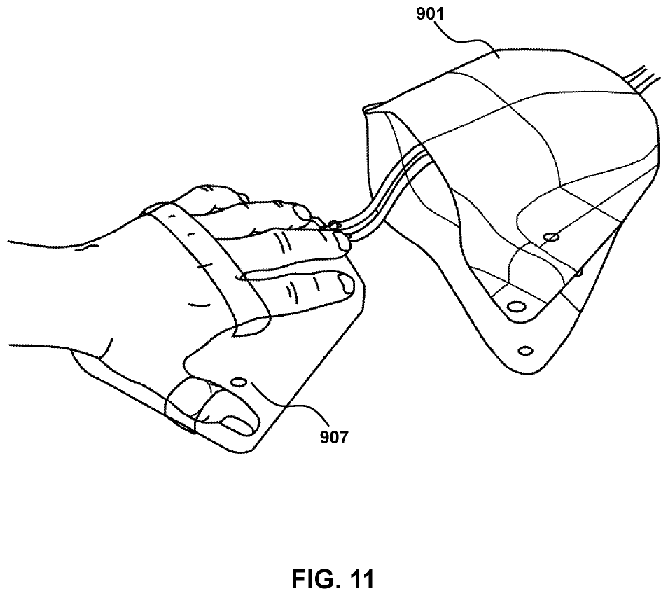

FIG. 11 is a perspective view of an embodiment of the bladder (907) removed from the housing (901) of the glabrous skin contact device. The bladder (907) can be removed from the housing (901) to facilitate cleaning of one or both of the bladder (907) and the housing (901). Upon completion of the cleaning, the bladder (907) and the housing (901) can be easily reattached.

In some embodiments, the glabrous skin contact device further comprises a means for creating negative pressure. In some embodiments, the means for creating negative pressure creates from about 0.5 to about 50 millimeters of mercury (mm-Hg) of vacuum. In some embodiments, the means for creating negative pressure creates from about 0.5 mm-Hg to about 5 mm-Hg of vacuum while in other embodiments, the means for creating negative pressure creates from about 3 mm-Hg to about 10 mm-Hg of vacuum, while in still other embodiments, the means for creating negative pressure creates from about 10 mm-Hg to about 25 mm-Hg of vacuum, and in still other embodiments, the means for creating negative pressure creates from about 15 mm-Hg to about 45 mm-Hg, and in some other embodiments, the means for creating negative pressure creates from about 25 mm-Hg to about 50 mm-Hg of vacuum. In some embodiments, the means for creating negative pressure creates from about 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 mm-Hg to about 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 mm-Hg of vacuum.

In some embodiments, the means for creating negative pressure can be disposed in an interior region of the glabrous skin contact device. In some embodiments, the means for creating negative pressure can be a vacuum pump. In such embodiments, the vacuum pump can be any conventional vacuum pump configured to fit with the glabrous skin contact device. In some embodiments, the vacuum pump is a diaphragm pump, a rotary vane pump, and the like. In some embodiments, the vacuum pump is portable and may advantageously be battery operated.

In other embodiments, the means for creating negative pressure comprises at least one of a vacuum pump, a fan, an air diverter, a compressed gas cartridge, or combinations thereof. In those embodiments involving compressed gas cartridges, the gas can be at least one of carbon dioxide, nitrous oxide, air, and combinations thereof and can have 5 psi (pounds per square inch) of pressure.

In some embodiments, the means for creating negative pressure can include fluid (such as water or air) flow through a small diameter orifice with a compensating diverter flow coming from the space to be evacuated.

In some embodiments, cyclonic conditions can provide the means for creating negative pressure. These cyclonic conditions can be created by using various fans, etc.

In some embodiments where the glabrous skin contact device includes a means for creating negative pressure, the means for creating negative pressure can be selectively operated. In some embodiments, selective operation can be achieved through the use of a vacuum pump switch operably connected to a vacuum pump wherein the vacuum pump switch is disposed on an exterior surface of the glabrous skin contact device. In some of these embodiments, selective operation enables operation of the means for creating negative pressure optionally, variably, and/or intermittently. Having this variety of selective operation settings allows a user of the glabrous skin contact device to deploy the means for creating negative pressure as desired or according to an intermittent setting, in response to a programmed control, in response to a sensor reading, etc. In this way, negative pressure can be applied to the glabrous skin most effectively rather than continuously.

Surprisingly, these inventors have found that when the user is heat stressed due to exertion, environment, or other factors, the vasculature of the glabrous region may be optimally dilated by such that vacuum offers little added benefit to cooling thus having the option to not use vacuum can help improve comfort and efficacy. And, under less heat stressed conditions the vacuum augments the cooling effect by optimally dilating the vascular bed of the glabrous skin and by opening the arterio venous anastomoses necessary for optimal reduction of the core body temperature. Thus, having the ability to optionally and selectively use vacuum offers the most effective and efficient way to modify body temperature.

In some embodiments, the glabrous skin contact unit further comprises biometric sensors. These biometric sensors can include vasoconstriction sensors, vasodilation sensors, body temperature sensors, and/or skin surface temperature sensors that can be used alone or in combination with each other. Those of skill in the art will be aware of conventional blood flow monitoring sensors that can serve as vasoconstriction and/or vasodilation sensors. Monitoring for vasoconstriction/vasodilation can be important in body temperature monitoring devices and methods because vasoconstriction/vasodilation of a glabrous skin surface in response to cold/warm stimuli can decrease the efficiency of body temperature lowering/raising. In some embodiments, the blood flow monitoring sensors for detecting vasoconstriction and/or vasodilation employ light sensors. In some embodiments, the blood flow monitoring sensors detect blood velocity, pulse rate and/or blood flow through distal appendages. In some embodiments, the body temperature and skin surface temperature sensors can include various temperature probes disposed in an internal region of the glabrous skin contact device. Any conventional temperature sensing device can be used. In some embodiments, an infrared thermometer can be used as a body temperature sensor. In some embodiments, electronic thermal probes may be used to sense the temperature of the inflow and outflow of the temperature transfer fluid or the surface temperature of the glabrous skin at both the arterial inflow or venous outflow from the glabrous skin appendage.

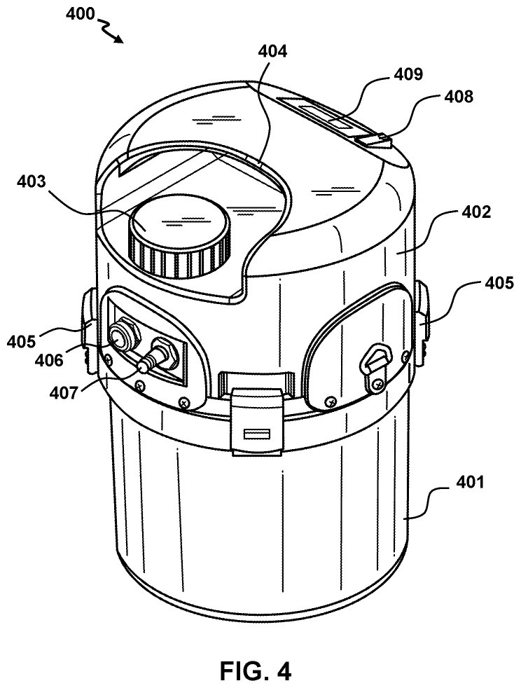

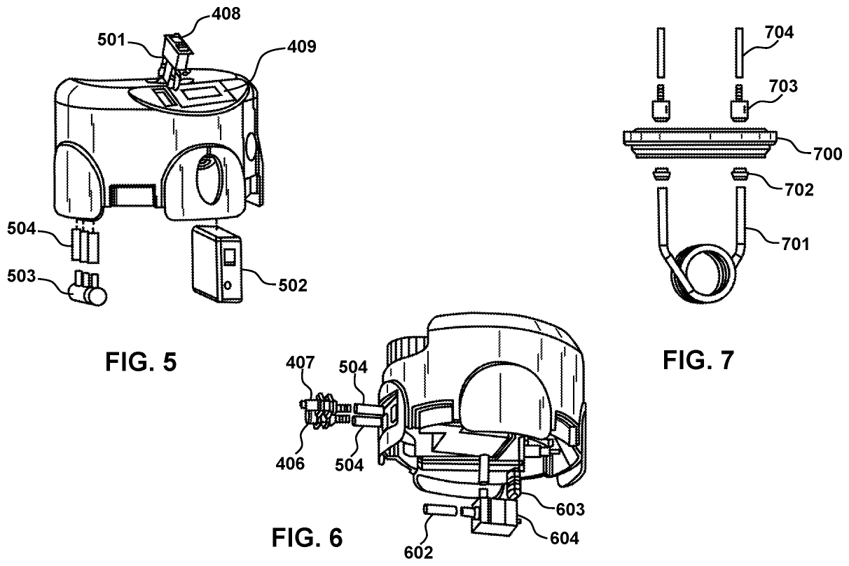

FIG. 4 is a perspective view of an embodiment of a heat exchange device (400) comprising an insulated container portion (401) for containing a heat sink material releasably connected to a control unit portion (402) wherein the control unit portion comprises a thermostatic mixing valve (shown as 503 in FIG. 5) disposed in an interior region of the control unit portion that maintains a temperature of a temperature transfer fluid in a range of from about 10 C to about 25 C. In some embodiments, the temperature of the temperature transfer fluid is from about 10 C, 11 C, 12 C, 13 C, 14 C, 15C, 16 C, 17 C, 18C, 19 C, 20 C, 21 C, 22 C, 23 C, or 24 C to about 11 C, 12 C, 13 C, 14 C, 15C, 16 C, 17 C, 18C, 19 C, 20 C, 21 C, 22 C, 23 C, 24 C, or 25 C.

In some embodiments, the heat exchange device also includes a port (403) that protrudes through an opening in the control unit portion. The port (403) provides a mechanism for easily adding the heat sink material to the heat exchange device without needing to separate the insulated container portion (401) from the control unit portion (402). In some embodiments, the insulated container portion (401) has an interior volume of from about 1 fluid quart to about 4 fluid quarts. In some embodiments, the insulated container portion (401) has an interior volume of from about 1, 1.5, 2, 2.5, 3, or 3.5 fluid quarts to about 1.5, 2, 2.5, 3, 3.5, or 4 fluid quarts.

In some embodiments, the heat sink material comprises at least one of a liquid, a gas, a solid, or combinations thereof. In some embodiments, the heat sink material comprises a mixture of water and ice.

In some embodiments, the insulated container portion has an R value of at least R-3, and preferably at least R-7, and even more preferably at least R-15. R value refers to a material's resistance to conductive heat flow and is measured or rated in terms of its thermal resistance. Using materials with these R values for the insulated container portion provides insulation for the heat sink material contained in the insulated container portion that enables the heat sink material to be less impacted by the external environment. In other words, the problem of excessive parasitic heat loss is avoided by using materials with an R value of at least R-3. Those of skill in the art will recognize that there are any number of materials that can provide these R values. Some examples include, but are not limited to, polystyrenes, polyisocyanurates, polyurethanes, urethanes, polyols, celluloses, fiber glasses, phenolics, vacuum space, and the like. In some embodiments, the insulated container portion can comprise a stainless steel, double-walled, vacuum canister.

Another convenience feature of the heat exchange device is the integrated grip feature (404). In some embodiments, the control unit portion has an exterior surface that includes an integrated grip feature (404). This integrated grip feature (404) provides a means for easily handling or carrying the heat exchange device (400). While the integrated grip feature shown in FIG. 4 shows an embedded handle, it will be appreciated that any other handling or carrying feature located on an exterior surface of the control unit portion will fall within the scope of these inventions. For example, in some embodiments, the integrated grip feature could include a D pull handle, a flush pull handle, a cup handle, a knob, and the like.

For releasably connecting the insulated container portion (401) to the control unit portion (402), in some embodiments, the heat exchange device (400) comprises at least one first attachment mechanism (405) that releasably attaches to a correspondingly mated at least one second attachment mechanism located on the control unit portion. In some embodiments, the at least one first and second attachment mechanisms comprise at least one of a series of threads for a screw attachment mechanism, a cam latch, a compression latch, a slam latch, a draw latch, a sliding latch, or combinations thereof.

For movement of the temperature transfer fluid in and out of the heat exchange device, in some embodiments, the heat exchange device (400), further comprises a female coupling (406) and a male coupling (407) both of which protrude through openings in the control unit portion and form passageways for the temperature transfer fluid. It will be appreciated that any suitable means for forming passageways for the temperature transfer fluid can be used including, but not limited to more than one female coupling, more than one male coupling, a single coupling, etc.

For operation of the heat exchange device, in some embodiments, the heat exchange device (400) further comprises a battery display (409) disposed on an exterior surface of the control unit portion, a power switch (408) disposed on an exterior surface of the control unit portion.

FIG. 5 is an exploded view of an embodiment of the control unit portion of the heat exchange device. This figure provides an alternative view of the battery display (409) and the power switch (408). In some embodiments, the power switch (408) is connected to a battery connector (501) which is connectedly attached to a battery (502). This allows a user to easily operate the control unit portion. While the illustrated embodiment shows the power switch to be a rocker switch, any type of conventional switch could be used instead.

Additionally, in some embodiments, the control unit portion further comprises a thermostatic mixing valve (503) and tubing units (504) for passage of the thermostatic mixing fluid.

In some embodiments, the thermostatic mixing valve (503) maintains a temperature of the temperature transfer fluid in a range of from about 10 C to about 25 C. In other embodiments, the temperature of the temperature transfer fluid is maintained in a range of from about 11 C to about 20 C, while in still other embodiments, the temperature of the temperature transfer fluid is maintained in a range of from about 12 C to about 19 C. In some embodiments, the temperature of the temperature transfer fluid is maintained in a range of from about 10 C, 10.5 C, 11 C, 11.5 C, 12 C, 12.5 C, 13 C, 13.5 C, 14 C, 14.5 C, 15C, 15.5 C, 16 C, 16.5 C, 17 C, 17.5 C, 18C, 18.5 C, 19 C, 19.5 C, 20 C, 20.5 C, 21 C, 21.5 C, 22 C, 22.5 C, 23 C, 23.5 C, 24 C, or 24.5 C to about 10.5 C, 11 C, 11.5 C, 12 C, 12.5 C, 13 C, 13.5 C, 14 C, 14.5 C, 15 C, 15.5 C, 16 C, 16.5 C, 17 C, 17.5 C, 18C, 18.5 C, 19 C, 19.5 C, 20 C, 20.5 C, 21 C, 21.5 C, 22 C, 22.5 C, 23 C, 23.5 C, 24 C, 24.5 C, or 25 C.

In some embodiments, the thermostatic mixing valve comprises at least one of a liquid-filled bellows, a wax bellows, a bi-metal coil, or combinations thereof. In some embodiments, the wax bellows includes a wax with a melting point of from about 10 C to about 20 C, and preferably at about 15 C.

In some embodiments, the thermostatic mixing valve comprises a piston and a spring. The piston and spring operate to move the temperature transfer fluid in response to its temperature.

FIG. 6 is another exploded view of an embodiment of the control unit portion of the heat exchange device. This view shows an embodiment of the control unit portion comprising a pump (604) with a splice wire (603) which allows power to flow from the battery to the pump and pump tubing units (602). In embodiments illustrated here, we also see another view of tubing units (504) and the female coupling (406) and male coupling (407).

In some embodiments, the pump is designed to maintain flow of a temperature transfer fluid through a patterned perfusion pad at a rate that approximates the rate of blood flow in the glabrous skin capillary bed and the arterio venous anastomoses. In some embodiments, the rate of flow for the temperature transfer fluid through the patterned perfusion pad is from about 3 liters per minute to about 7 liters per minutes. In some embodiments, the rate of flow is from about 3 liters, 3.5 liters, 4 liters, 4.5 liters, 5 liters, 5.5 liters, 6 liters, or 6.5 liters per minute to about 3.5 liters, 4 liters, 4.5 liters, 5 liters, 5.5 liters, 6 liters, 6.5 liters, or 7 liters per minute.

FIG. 7 is an exploded view of an embodiment of a gasket (700) in the heat exchange device. In some embodiments, the heat exchange device further comprises a gasket (700) positioned between the insulated container portion and the control unit portion having an upper surface facing the control unit portion and a lower surface facing the insulated portion. As such, this gasket forms an interface between the control unit portion and the insulated portion of the heat exchange device. In some embodiments, the heat exchange device further comprises a heat exchanger (701) that is attached to bushings (702) that pass through openings in the gasket (700) where the bushings (702) attach to adapters (703) which in turn attach to heat exchange tubing units (704). The heat exchanger (701) is disposed in an interior region of the control unit portion and contacts the heat sink material housed in an interior region of the insulated portion.

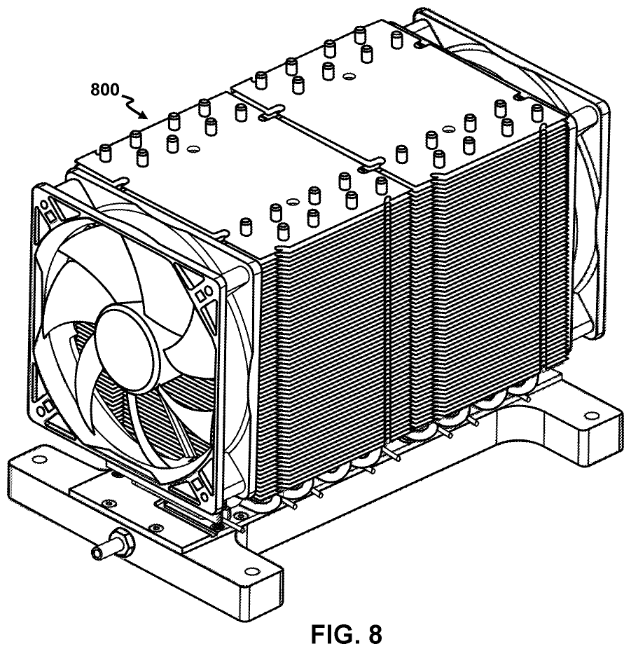

FIG. 8 is a perspective view of an embodiment of the heat exchanger. The heat exchanger comprises Peltier elements (801).

In some embodiments, tubing units located in an interior region of the control unit portion create a closed loop system where the temperature transfer fluid flows from a reservoir through the pump to a split where the temperature transfer fluid flows to either the heat exchanger or the thermostatic mixing valve. Temperature transfer fluid that flows through the heat exchanger is either warmed or cooled depending on the temperature of the heat sink material into which the heat exchanger is submerged. The warmed or cooled temperature transfer fluid then flows to the thermostatic mixing valve where its temperature is adjusted and then the temperature adjusted temperature transfer fluid flows into and through the bladder. Temperature transfer fluid that flows to the thermostatic mixing valve at the split after the pump is used to obtain the desired temperature. In some embodiments, this closed loop system with the split after the pump creates an efficient system that only requires a single pump. In some embodiments, the heat exchanger is a metal such as copper whose thermal conductivity enables rapid temperature transfer from the heat sink material to the temperature transfer fluid.

In other embodiments, the control unit does not comprise a heat exchanger submerged in a heat sink material. In such embodiments, the temperature transfer fluid is contained in the insulated portion and pumped through the thermostatic mixing valve and into the bladder in a loop without involving a heat exchanger. In some non-heat exchanger embodiments, more than one pump can be used.

In some embodiments, the temperature transfer fluid comprises at least one of a liquid, a gas, a solid, or combinations thereof. In some embodiments, the temperature transfer fluid comprises a mixture of ice and water.

In some embodiments, the heat sink material comprises at least one of a liquid, a gas, a solid, or combinations thereof. In some embodiments, the heat sink material comprises a mixture of ice and water.

In some embodiments, the heat exchange device is adapted to be used together with the glabrous skin contact device. In these embodiments, a body temperature modifying device comprises a heat exchange unit connected by at least one tubing unit to a glabrous skin contact unit, wherein the glabrous skin contact unit comprises a housing enclosing a bladder disposed in an interior region of the glabrous skin contact unit, and wherein the housing has a molded bottom surface and a contoured upper surface. As an overview, in these embodiments, the at least one tubing unit connects to the inlet and/or outlet ports on the glabrous skin contact unit and to the male and/or female couplings on the heat exchange unit. In operation, the temperature transfer fluid flows through the at least one tubing unit into the bladder housed in the interior of the glabrous skin contact unit. A user of the device inserts an appendage into the opening of the glabrous skin contact unit where the bladder is resting on the protuberance formed into the molded bottom surface of the glabrous skin contact unit thus modifying the user's body temperature by enabling heat transfer from the temperature transfer fluid in the bladder and the user's glabrous skin. Because the body temperature modifying device combines the glabrous skin contact device and the heat exchange device, features of those devices as described previously will be understood to be features of the body temperature modifying device.

For example, as with the glabrous skin contact device, the glabrous skin contact unit in some embodiments of the body temperature modifying device has a molded bottom surface that includes a protuberance that protrudes into an interior region of the glabrous skin contact unit. In addition to the protuberance, in some embodiments of the body temperature modifying device, the glabrous skin contact unit has a Young's Modulus that is greater than a Young's Modulus of the contoured upper surface. In some of these embodiments, the Young's Modulus of the molded bottom surface of the skin contact unit is from about 0.75 GPa to about 4.15 GPa.

As with the glabrous skin contact device, the bladder of this glabrous skin contact unit in some embodiments of the body temperature modifying device has a thermal conductivity of 0.1 to about 0.5 watts per meter-kelvin (W·m−1·K−1).

In some embodiments, the housing provides sufficient insulating capacity such that parasitic heat loss from the bladder to the environment is minimized. In some embodiments, the insulating capacity of the housing is from about R-0.01 to about R-0.1. Without this insulating capacity, efficiency and efficacy of the body temperature modifying device are diminished.

Also, as with the glabrous skin contact device, the glabrous skin contact unit in some embodiments of the body temperature modifying device further comprises a means for creating negative pressure. In some embodiments, the amount of negative pressure created can be from about 0.5 mm-Hg to about 50 mm-Hg of vacuum. In some embodiments, the means for creating negative pressure created can be selectively operated.

As with the heat exchange device, in some embodiments, the heat exchange unit of the body temperature modifying device further comprises an insulated portion and a control unit portion. And, in some embodiments of the body temperature modifying device, the control unit portion of the heat exchange unit comprises a thermostatic mixing valve disposed in an interior region of the control unit portion that maintains a temperature of a temperature transfer fluid in a range of from about 10 C to about 25 C. In some embodiments, the thermostatic mixing valve of the body temperature modifying device comprises at least one of a liquid-filled bellows, a wax bellows, a bi-metal coil, or combinations thereof. In embodiments where the thermostatic mixing valve comprises a wax bellows, the wax bellows comprises a wax with a melting point of from about 10 C to about 20 C.

As with the heat exchange device, in some embodiments of the body temperature modifying device, the insulated portion of the heat exchange unit has an R-value of at least R-3.

In some embodiments, the at least one tubing unit of the body temperature monitoring device comprises a first section of tubing is connected to an inlet port located on one end of the bladder and a second section of tubing is connected to an outlet port located on the same end of the bladder as the first section of tubing.

In addition to the body temperature modifying device described above, in some embodiments a body temperature modifying device comprises a body temperature modifying unit and a power source, wherein the body temperature modifying unit comprises a glabrous skin contact surface and a thermoelectric temperature modifying unit.

In some of these embodiments, the body temperature modifying units comprise Peltier elements. Thermoelectric temperature modifying units can have certain advantages such as rapid temperature change and ease of use. As a result, body temperature modifying devices using thermoelectric temperature modification can offer these advantages in athletic, occupational, and medical situations.

In some of these embodiments, the glabrous skin contact surface includes a protuberance. This protuberance can enhance the users experience and provide effective and efficient body temperature modification. In some embodiments, the protuberance has a height of from about 1 mm to about 100 mm at its apex. In some embodiments, the protuberance has a width of from about 12 cm to about 15 cm and a length along its long axis of from about 18 cm to about 23 cm.

Additionally, in some of these embodiments, the body temperature modifying device further comprises a means for creating negative pressure. In some embodiments, the means for creating negative pressure creates from about 0.5 mm-Hg to about 50 mm-Hg. Additionally, in some embodiments, the means for creating negative pressure can be selectively operated.

Other devices developed by these inventors include a multi-appendage device comprising a plurality of a heat exchange units connected by a plurality of tubing units to at least two glabrous skin contact units, wherein each of the at least two glabrous skin contact units comprises a housing enclosing a bladder, and wherein the housing has a molded bottom surface and a contoured upper surface. In some embodiments, the molded bottom surface comprises a protuberance that protrudes into an interior region of the glabrous skin contact unit. Having a device with a plurality of units can be useful in group situations. These group situations can include, but are not limited to, fitness centers, work sites, athletic competitions, and the like. A multi-appendage device can also be useful for a single user who desires body temperature modification involving at least two appendages.

In some embodiments, the plurality of heat exchange units, tubing units, and the at least two glabrous skin contact units includes from about 2 to about 30 units. In other embodiments, the plurality of heat exchange units can be from about 2 to about 5 and the at least two glabrous skin contact units can be from about 2 to about 30. In some embodiments, one heat exchange unit can be connected by tubing units to several glabrous skin contact units thereby making efficient use of the heat exchange units. For improved operations, in some embodiments, the device further comprises a cabinet that houses the plurality of heat exchange units, tubing units, and the at least two glabrous skin contact units.

In some alternative multi-appendage devices, the multi-appendage device accommodates one or more users and comprises at least two body temperature modifying units and a power source, wherein the at least two body temperature modifying units comprise a glabrous skin contact surface. In some embodiments of these devices, the body temperature modifying units comprise thermoelectric temperature modifying units, and in some embodiments, thermoelectric temperature modifying units comprise Peltier elements. In some embodiments, the power source can include at least one of electricity, battery, solar, nuclear, or combinations thereof.

In some embodiments, the glabrous skin contact surface comprises a layer of thermally conductive material that is placed in contact with glabrous skin on one side and the thermoelectric modifying unit on the other side. In some embodiments, this layer of thermally conductive material can improve the user's comfort and in some embodiments, the layer of thermally conductive material can be disposable.

In some embodiments, the glabrous skin contact surface is movable to allow for postural adjustments by the mammalian user which can improve comfort. In some embodiments, the movable glabrous skin contact surface is a rolling ball configuration.

Those of skill in the art will be aware of the various means for providing thermoelectric temperature modifications. These means for providing thermoelectric temperature modifications operate by using electrical current to pull heat from one side of the unit to the other resulting in one side getting cooler while the other side gets hotter. These means can be configured using various modules that meet the needs of the system.

In some embodiments, the multi-appendage device further comprises a cabinet that houses the body temperature modifying units and the electrical power source. In some embodiments, the multi-appendage device can be movable and/or portable.

In some embodiments, a system comprises an athletic equipment component and a temperature adjustment component spatially located within a range of each other wherein the temperature adjustment component comprises a glabrous skin contact device wherein the glabrous skin contact device comprises a housing enclosing a bladder and wherein the housing has a molded bottom surface and a contoured upper surface. In some embodiments, the range is a distance of from about zero to about 100 feet. For example, the system could include at least one of a stationary bicycle or rowing machine as the athletic equipment and the glabrous skin contact device of the temperature adjustment component could be located on a handlebar or foot pedal of the athletic equipment. Similarly, a treadmill, elliptical trainer, or stair stepper could be configured to incorporate the glabrous skin contact device into a handrail. Alternatively, the system could include a support that houses weights, resistance equipment, etc. where the glabrous skin contact device could be located on the support (fixedly or removably) so that the user can access the glabrous skin contact device as needed while using the athletic equipment or during rest periods.

In some embodiments, the temperature adjustment component of the system further comprises a heat exchange device comprising an insulated container portion for containing a heat sink material releasably connected to a control unit portion wherein the control unit portion comprises a thermostatic mixing valve disposed in an interior region of the control unit portion that maintains a temperature of a temperature transfer fluid in a range of from about 10 C to about 25 C, and wherein the heat exchange device is connected to the glabrous skin contact device by at least one tubing unit that enables the temperature transfer fluid to flow into the bladder.

In addition to the various inventive devices described here, these inventors have also invented methods. In some embodiments, a method of manipulating mammalian body temperature comprises the steps of subjecting a glabrous skin surface to a body temperature modifying device comprising a heat exchange unit connected by at least one tubing unit to a glabrous skin contact unit, wherein the glabrous skin contact unit comprises a housing enclosing a bladder disposed in an interior region of the glabrous skin contact unit, and wherein the housing has a molded bottom surface and a contoured upper surface, and operating the heat exchange unit with a temperature transfer fluid. In some embodiments, the shape of the glabrous skin contact unit conforms to the anatomy of an appendage with the mammal's glabrous skin. In some embodiments, the molded bottom surface includes a protuberance that protrudes in an interior region of the glabrous skin contact unit. In some embodiments of these methods, the method further comprises a step of cooling the temperature transfer fluid while in other embodiments, the method further comprises a step of warming the temperature transfer fluid. By cooling the temperature transfer fluid, mammalian body temperature can be lowered and by warming the temperature transfer fluid, mammalian body temperature can be raised.

These methods can be practiced in situations where either an increase in body temperature is desired, a decrease in body temperature is desired, or maintenance of body temperature is desired. These situations can include, but are not limited to, surgical or medical situations where maintaining a particular body temperature is desired or an increase in body temperature is desired, athletic situations where maintenance or a decrease in body temperature is desired, and/or occupational situations like firefighting, mining, emergency services, etc. where body temperature management could require the ability for either maintaining, increasing, or decreasing body temperature. The temperature of the temperature transfer fluid that flows from the heat exchange unit to the bladder disposed in the interior region of the skin contact unit can be increased to cause an increase in body temperature or decreased to cause a decrease in body temperature. The molded bottom surface of the glabrous skin contact unit, which can have a protuberance in some embodiments, provides an effective way to manipulate body temperature.

In some embodiments, the mammal involved in the method is a human, while in other embodiments, the mammal can include an animal such as a cat, a dog, a pig, a rat, a mouse, a rabbit, and the like.

Another method developed by these inventors is a method of improving athletic performance. In some embodiments, the method comprises the steps of subjecting a glabrous skin surface to a glabrous skin contact device wherein the glabrous skin contact device comprises a molded bottom surface, a contoured upper surface, and a bladder disposed in an interior region of the glabrous skin contact device, and wherein the glabrous skin surface is subjected to the glabrous skin contact device for a temperature adjustment time of at least fifteen seconds.

In some embodiments, the method of improving athletic performance further comprises connecting the glabrous skin contact device to a heat exchange device comprising an insulated container portion for containing a heat sink material releasably connected to a control unit portion, wherein the control unit portion comprises a thermostatic mixing valve disposed in an interior region of the control unit portion that maintains a temperature of a temperature transfer fluid in a range of from about 10 C to about 25 C, and wherein the connecting step enables the temperature transfer fluid to flow into the bladder.

In some embodiments, the temperature adjustment time is in a range of from about 15 seconds to about 10 minutes. In some embodiments, the method of improving athletic performance involves interspersing the temperature adjustments with athletic activity while in other embodiments, the temperature adjustment occurs simultaneously with the athletic activity. For example, weightlifting athletic performance can be improved by interspersing temperature adjustments with sets of weightlifting. For another example, stationary bicycle athletic performance can be improved by simultaneously and continuously conducting temperature adjustments according to the method of improving athletic performance. In some embodiments, the temperature transfer fluid is cooled to a temperature of from about 10 C to about 25 C. In other embodiments, the temperature transfer fluid is cooled to a temperature of from about 10 C, 11 C, 12 C, 13 C, 14 C, 15C, 16 C, 17 C, 18C, 19 C, 20 C, 21 C, 22 C, 23 C, or 24 C to about 11 C, 12 C, 13 C, 14 C, 15C, 16 C, 17 C, 18C, 19 C, 20 C, 21 C, 22 C, 23 C, 24 C, or 25 C.

Additionally, these inventors have developed athletic performance processes. In some embodiments, an interval athletic performance process comprises the steps of completing an athletic performance act, conducting a temperature adjustment, and repeating the athletic performance act and temperature adjustment serially for a number of repetitions of from about 2 to about 15.

In some embodiments, the temperature adjustment step of the interval athletic process further comprises the steps of subjecting a glabrous skin surface to a glabrous skin contact device comprising a molded bottom surface, a contoured upper surface, and a bladder disposed in an interior region of the glabrous skin contact device, and wherein the glabrous skin surface is subjected to the glabrous skin contact device for a temperature adjustment time of from about 15 seconds to about 5 minutes. In some embodiments, the temperature adjustment time is from about 1, 1.5, 2, 2.5, 3, 3.5, 4, or 4.5 minutes to about 1.5, 2, 2.5, 3, 3.5, 4, 4.5, or 5 minutes.

In some embodiments, the interval athletic performance process further comprises connecting the glabrous skin contact device to a heat exchange device comprising an insulated container portion for containing a heat sink material releasably connected to a control unit portion, wherein the control unit portion comprises a thermostatic mixing valve disposed in an interior region of the control unit portion that maintains a temperature of a temperature transfer fluid in a range of from about 10 C to about 25 C, and wherein the connecting step enables the temperature transfer fluid to flow into the bladder.

In some embodiments, the temperature transfer fluid is cooled to a temperature of from about 10 C, 11 C, 12 C, 13 C, 14 C, 15C, 16 C, 17 C, 18C, 19 C, 20 C, 21 C, 22 C, 23 C, or 24 C to about 11 C, 12 C, 13 C, 14 C, 15C, 16 C, 17 C, 18C, 19 C, 20 C, 21 C, 22 C, 23 C, 24 C, or 25 C.

In some embodiments, this interval athletic performance process with the temperature adjustment step allows the athlete to complete a greater number of repetitions than they could without the temperature adjustment step. This athletic performance process can benefit athletes involved in a wide range of athletic activities. These athletic activities can include, but are not limited to, weightlifting, pull ups, push-ups, bench pressing, leg pressing, dead lift, aerobic training (including but not limited to steady-state cardio, zone training, sprint intervals, etc.), circuit training, high intensity interval training (HIIT), cross training, plyometrics, isometrics, and competition sports. In some embodiments, the athletic performance act comprises at least one of weightlifting, pull ups, pushups, bench pressing, leg pressing, dead lift, aerobic training, circuit training, high intensity interval training, cross training, or combinations thereof.

In addition to the interval athletic process, these inventors have also developed continuous athletic processes. In some embodiments, a continuous athletic performance process comprises the steps of conducting an athletic performance act and simultaneously conducting a temperature adjust act, wherein the athletic performance act has an improved performance metric compared to the athletic performance act conducted without the temperature adjustment step.

In some continuous athletic processes, the improved performance metric comprises at least one of duration, exertion, heart rate, strength, perceived exertion, decision making, or combinations thereof.

In some embodiments of the continuous athletic process, the temperature adjustment step further comprises the step of subjecting a glabrous skin surface to a glabrous skin contact device comprising a molded bottom surface, a contoured upper surface, and a bladder disposed in an interior region of the glabrous skin contact unit.

In other continuous athletic processes, the athletic performance process further comprises connecting the glabrous skin contact device to a heat exchange device comprising an insulated container portion for containing a heat sink material releasably connected to a control unit portion wherein the control unit portion comprises a thermostatic mixing valve disposed in an interior region of the control unit portion that maintains a temperature of a temperature transfer fluid in a range of from about 10 C to about 25 C, and wherein the connecting step enables the temperature transfer fluid to flow into the bladder. In some embodiments, the temperature transfer fluid is cooled to a temperature of from about 10 C, 11 C, 12 C, 13 C, 14 C, 15C, 16 C, 17 C, 18C, 19 C, 20 C, 21 C, 22 C, 23 C, or 24 C to about 11 C, 12 C, 13 C, 14 C, 15C, 16 C, 17 C, 18C, 19 C, 20 C, 21 C, 22 C, 23 C, 24 C, or 25 C.

In some embodiments, the glabrous skin contact device is in communication with a mobile device (not shown) to provide information regarding the status of the glabrous skin contact device. For example, the glabrous skin contact device can notify the mobile device that the glabrous skin contact device is ready for use. Further, it can track the temperature, the cooling or heating time remaining, and even the battery life of the heat exchange device (in addition to the battery display (409)).

Additionally, the mobile device connected to the glabrous skin contact device can alert the user when the temperature adjustment time is complete. The timer starts when the glabrous skin surface is inserted into the glabrous skin contact device. Upon completion of the temperature adjustment time (about 15 seconds to about 5 minutes), the mobile device will beep or otherwise notify the user to remove the glabrous skin surface.

The mobile device is connected to an app that communicates with the glabrous skin contact device. In embodiments, the mobile device can also track the user's workout or activity. The mobile device can be connected to the glabrous skin contact device through a Bluetooth or other wireless connection. Alternatively, the connection can be wired.

It is to be understood that these inventions are not limited to the particular embodiments described. It is also to be understood that the terminology used herein is for the purpose of describing particular embodiments only, and is not intended to be limiting, since the scope of these inventions will be limited only by the appended claims.

Where a range of values is provided, it is understood that each intervening value is included, between the upper and lower limit of that range and any other stated or intervening value in that stated range, is encompassed within the invention, unless the context clearly dictates otherwise. The upper and lower limits of any smaller ranges may independently be included in the smaller ranges and are also encompassed within the inventions, subject to any specifically excluded limit in the stated range. Where the stated range includes one or both of the upper and lower limits, ranges excluding either or both of those included limits are also included in the invention.

Certain ranges may be presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present inventions, representative illustrative methods and materials are herein described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present inventions are not entitled to antedate such publication by virtue of prior invention. Further, the dates of publication provided may be different from the actual publication dates which may need to be independently confirmed.

It is noted that, as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation.

Embodiments of the devices and their specific spatial characteristics and/or abilities include those shown or substantially shown in the drawings or which are reasonably inferable from the drawings. Such characteristics include, for example, one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal; distal), and/or numbers (e.g., three surfaces; four surfaces), or any combinations thereof. Such spatial characteristics also include, for example, the lack (e.g., specific absence of) one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal), and/or numbers (e.g., three surfaces), or any combinations thereof.

As will be apparent to those of skill in the art upon reading this disclosure, each of the individual embodiments described and illustrated herein has discrete components and features which may be readily separated from or combined with the features of any of the other several embodiments without departing from the scope or spirit of the present inventions. Any recited method can be carried out in the order of events recited or in any other order which is logically possible.

### EXAMPLES

### Example 1: Fireman's Carry Protocol

To understand the recovery benefit from using a body temperature modifying device in between intense rounds of exercise, a volunteer subject performed three sessions of the Fireman's Carry Protocol where two of the sessions did not involve the body temperature modifying device and one session involved the body temperature modifying device.

The body temperature modifying device that was used included a heat exchange unit connected by tubing to a glabrous skin contact unit. The heat exchange unit included an insulated portion with an R-value of R-5 that held 2 quarts of crushed ice and water that served as the temperature adjustment fluid. The heat exchange unit also included a thermostatic mixing valve that maintained the temperature of the temperature adjustment fluid at a temperature of about 15 C. The glabrous skin contact unit included a housing with a molded bottom surface having an inward-facing protuberance on top of which sat a bladder made of thermoplastic polyurethane. The cooled water flowed from the insulated portion of the heat exchange unit through the tubing and into the bladder which was inside the glabrous skin contact unit. The subject inserted their hand into the glabrous skin contact unit where the palm of their hand made contact with the bladder.

The Fireman's Carry protocol involves having the subject wear an 80 lb. weight vest while walking on a treadmill set at a 15% incline at a pace of 2.5-3.5 mile per hour. The speed is adjusted to sustain heart rate at 83-87% of the maximum heart rate for the subject. The protocol involves having the subject walk on the treadmill for 15 minutes then rest for 5 minutes off the treadmill and then walk again for another 15 minutes.

Results of the sessions are shown in Table 1. The data were collected by having the subject complete a questionnaire. For the session where the body temperature modifying device was used, the subject used the device for the entirety of the protocol (i.e. for 35 minutes).

TABLE 1
Results from Fireman's Carry Sessions

| | Session 1 - | Session 2 - | Session 3 - |
|---|---|---|---|
| | no body | no body | body |
| | temperature | temperature | temperature |
| | modifying | modifying | modifying |
| Measure | device | device | device |
| Rate of perceived | 7 out of 10 | 7 out of 10 | 5 out of 10 |
| exertion (RPE) | | | |
| Time to total energy | 3 minutes | 3 minutes | 1 minute |
| recovery during the | 47 seconds | 33 seconds | 34 seconds |
| 5 minute rest period | | | |
| Time to total | 3 minutes | 3 minutes | 1 minute |
| recovery of baseline | 47 seconds | 33 seconds | 34 seconds |
| respiration rate | | | |

These results show that using the body temperature modifying device increased the subject's ability to recover from an intense workout.

What is claimed is:

1. A body temperature modifying device comprising a heat exchange unit connected by at least one tubing unit to a glabrous skin contact unit, wherein the glabrous skin contact unit comprises a housing enclosing a bladder disposed in an interior region of the glabrous skin contact unit, and wherein the housing has a molded bottom surface and a contoured upper surface.

2. The body temperature modifying device as in claim 1, wherein the molded bottom surface includes a protuberance that protrudes into an interior region of the glabrous skin contact unit.

3. The body temperature modifying device as in claim 1, wherein the molded bottom surface has a Young's Modulus that is greater than a Young's Modulus of the contoured upper surface.

4. The body temperature modifying device as in claim 3, wherein the Young's Modulus of the molded bottom surface is from about 0.75 GPa to about 4.15 GPa.

5. The body temperature modifying device as in claim 1, wherein the bladder has a thermal conductivity of from about 0.1 to about 0.5 watts per meter-kelvin (W·m−1·K−1).

6. The body temperature modifying device as in claim 1, wherein the housing comprises at least one opening and the contoured upper surface comprises at least two passive closure features located on at least two edges of the at least one opening.

7. The body temperature modifying device as in claim 1, wherein the glabrous skin contact unit further comprises a means for creating negative pressure.

8. The body temperature modifying device as in claim 7, wherein the means for creating negative pressure creates from about 0.5 mm-Hg to about 50 mm-Hg of pressure.

9. The body temperature modifying device as in claim 7, wherein the means for creating negative pressure can be selectively operated.

10. The body temperature modifying device as in claim 1, wherein the at least one tubing unit comprises a first section of tubing and a second section of tubing, and wherein the first section of tubing is connected to an inlet port located on one end of the bladder and the second section of tubing is connected to an outlet port located on the same end of the bladder as the first section of tubing.

11. The body temperature modifying device as in claim 1, wherein the heat exchange unit further comprises an insulated portion and a control unit portion.

12. The body temperature modifying device as in claim 11, wherein the control unit portion comprises a thermostatic mixing valve disposed in an interior region of the control unit portion that maintains a temperature of a temperature transfer fluid in a range of from about 10 C to about 25 C.

13. The body temperature modifying device as in claim 12, wherein the thermostatic mixing valve comprises at least one of a liquid-filled bellows, a wax bellows, a bi-metal coil, or combinations thereof.

14. The body temperature modifying device as in claim 13, wherein the wax bellows comprises a wax with a melting point of from about 10 C to about 20 C.

15. The body temperature modifying device as in claim 11, wherein the insulated portion has an R-value of at least R-3.

16. The body temperature modifying device as in claim 1, wherein the heat exchange unit further comprises a heat exchanger disposed in an interior region of the control unit portion.

17. The body temperature modifying device as in claim 16, wherein the heat exchanger comprises Peltier elements.

18. The body temperature modifying device as in claim 1, wherein the bladder comprises a top surface that includes a plurality of protruding spots.

19. The body temperature modifying device as in claim 1, wherein the bladder comprises a top surface having straps to receive a glabrous skin surface.

20. The body temperature modifying device as in claim 1, wherein the bladder is removable from the housing to facilitate cleaning.

21. The body temperature modifying device as in claim 1, wherein the body temperature modifying device is in communication with a mobile device to provide information regarding the status of the body temperature modifying device.

22. A method of manipulating mammalian body temperature comprising the steps of subjecting a glabrous skin surface to a body temperature modifying device comprising a heat exchange unit connected by at least one tubing unit to a glabrous skin contact unit, wherein the glabrous skin contact unit comprises a housing enclosing a bladder disposed in an interior region of the glabrous skin contact unit, and wherein the housing has a molded bottom surface and a contoured upper surface, and operating the heat exchange unit with a temperature transfer fluid.

23. The method as in claim 22, wherein the molded bottom surface includes a protuberance that protrudes in an interior region of the glabrous skin contact unit.

24. The method as in claim 22, wherein the glabrous skin contact unit further comprises a means for creating negative pressure.

25. The method as in claim 22, further comprising a step of cooling the temperature transfer fluid.

26. The method as in claim 22, further comprising the step of warming the temperature transfer fluid.

27. The method as in claim 22, wherein the heat exchange unit includes Peltier elements.

28. The method as in claim 22, wherein the bladder comprises a top surface that includes a plurality of protruding spots.

29. The method as in claim 22, wherein the bladder comprises a top surface having straps to receive the glabrous skin surface.

30. The method as in claim 22, wherein the bladder is removable from the housing to facilitate cleaning.

31. The method as in claim 22, wherein the body temperature modifying device is in communication with a mobile device to provide information regarding the status of the body temperature modifying device.

32. A method of improving athletic performance comprising the steps of subjecting a glabrous skin surface to a glabrous skin contact device, wherein the glabrous skin contact unit comprises a molded bottom surface, a contoured upper surface, and a bladder disposed in an interior region of the glabrous skin contact unit, and wherein the glabrous skin surface is subjected to the glabrous skin contact device for a temperature adjustment time of at least 15 seconds.

33. The method as in claim 32, further comprising connecting the glabrous skin contact device to a heat exchange device comprising an insulated container portion for containing a heat sink material releasably connected to a control unit portion wherein the control unit portion comprises a thermostatic mixing valve disposed in an interior region of the control unit portion that maintains a temperature of a temperature transfer fluid in a range of from about 10 C to about 25 C, and wherein the connecting step enables the temperature transfer fluid to flow into the bladder.

34. The method as in claim 33, wherein the heat exchange device includes Peltier elements.

35. The method as in claim 32, wherein the bladder comprises a top surface that includes a plurality of protruding spots.

36. The method as in claim 32, wherein the bladder comprises a top surface having straps to receive the glabrous skin surface.

37. The method as in claim 32, wherein the bladder is removable from the housing to facilitate cleaning.

38. The method as in claim 32, wherein the glabrous skin contact device is in communication with a mobile device to provide information regarding the status of the glabrous skin contact device.
